(19) United States
(12) Patent Application Publication
(10) Pub. No.: US 2009/0157009 A1
(43) Pub. Date: Jun. 18, 2009

(54) Needle and cover device for a safety syringe

(76) Inventors: Wang-Ken Lee, Marietta, GA (US); Yi-Wei Lin, Yonghe City (TW)

Correspondence Address: PATENT, COPYRIGHT & TRADEMARK LAW GROUP, 4199 Kinross Lakes Parkway, Suite 275, RICHFIELD, OH, 44286, US

(21) Appl. No.: 12/000,797

(22) Filed: Dec. 18, 2007

(51) Int. Cl. A61M 5/32

(52) U.S. Cl. 604/192

(57) ABSTRACT

A needle and cover device is mounted detachably on a barrel with a plunger to form a safety syringe and has a needle member and a cover member. The needle member is mounted on the barrel and has a needle hub and a needle. The needle hub has a cylinder, multiple spokes and a needle mount. The spokes are formed on the cylinder and each spoke has a locking recess. The needle is mounted on the needle mount. The cover member is capable of locking and holding the needle member and has a locking ring and a cover. The locking ring has multiple locking protrusions formed on the locking ring and selectively engaged respectively with the locking recesses in the needle hub. When the cover member locks the needle member, pulling the cover member detaches the needle member from the barrel.

### BACKGROUND OF THE INVENTION

1. Field of the Invention

The present invention relates to a needle and cover device, and more particularly to a needle and cover device for a safety syringe that is mounted on a syringe and has a needle member and a cover member being capable of safely removing the needle member after the use of the needle member.

2. Description of Related Art

Syringes are common medical articles and have a barrel, a plunger and a needle hub. The needle hub has a needle. To prevent contagious diseases, the needles of the syringes and even the hollow barrels and plungers, are prohibited from reuse and must be discarded immediately after use. To keep nurses, doctors or workers dealing with discarded syringes from getting injured or infected by the used needles, safety syringes has been developed.

However, conventional safety syringes often have complicated structures and inconvenient operation.

To overcome the shortcomings, the present invention provides a needle and cover device to mitigate or obviate the aforementioned problems.

### SUMMARY OF THE INVENTION

The main objective of the invention is to provide a needle and cover device for a safety syringe that is mounted on a syringe and has a needle and a cover being capable of safely removing the needle after the use of the needle.

A needle and cover device is mounted detachably on a barrel with a plunger to form a safety syringe and has a needle member and a cover member. The needle member is mounted on the barrel and has a needle hub and a needle. The needle hub has a cylinder, multiple spokes and a needle mount. The spokes are formed on the cylinder and each spoke has a locking recess. The needle is mounted on the needle mount. The cover member is capable of locking and holding the needle member and has a locking ring and a cover. The locking ring has multiple locking protrusions formed on the locking ring and selectively engaged respectively with the locking recesses in the needle hub. When the cover member locks the needle member, pulling the cover member detaches the needle member from the barrel.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a needle and cover device for a safety syringe;

FIG. 2 is an exploded perspective view of the needle and cover device in FIG. 1;

FIG. 3 is a top view of the needle and cover device in FIG. 1 showing that the needle hub of the needle member extends in the mounting hole in the locking ring of the cover member;

FIG. 4 is an operational top view of the needle and cover device in FIG. 3 showing that the needle hub rotates in the central hole of the locking ring and locked by the locking ring;

FIG. 5 is a side view in partial section of the needle and cover device along line 5-5 in FIG. 4;

FIG. 6 is an operational side view in partial section of the needle and cover device in FIG. 5 mounted to a large barrel to form a safety syringe; and

FIG. 7 is an operational side view in partial section of the needle and cover device in FIG. 5 mounted in a small barrel to form a safety syringe.

### DETAILED DESCRIPTION OF THE PREFERRED EMBODIMENT

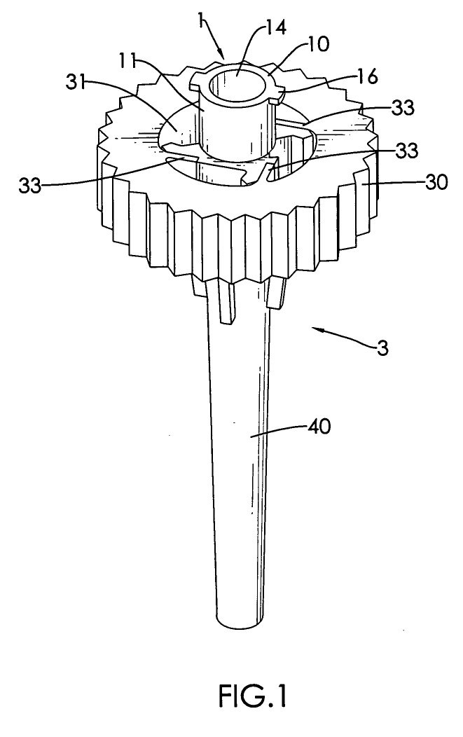

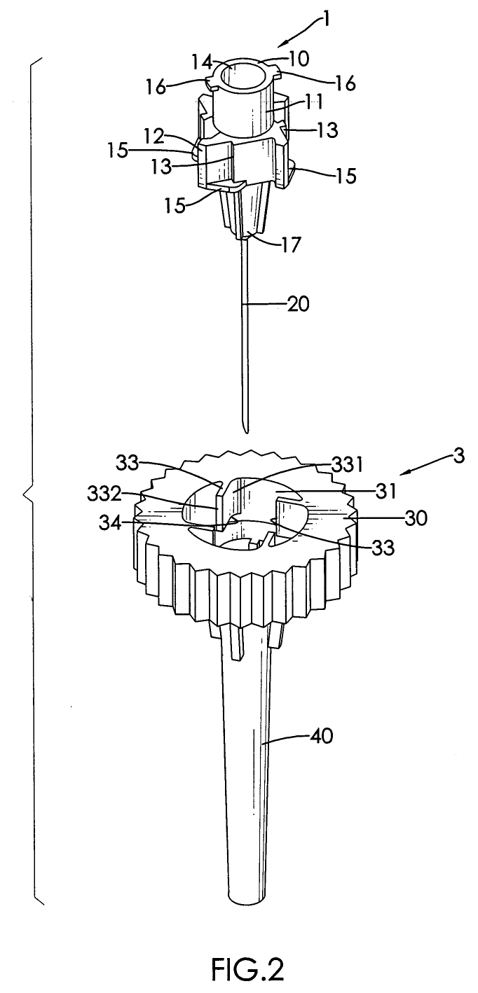

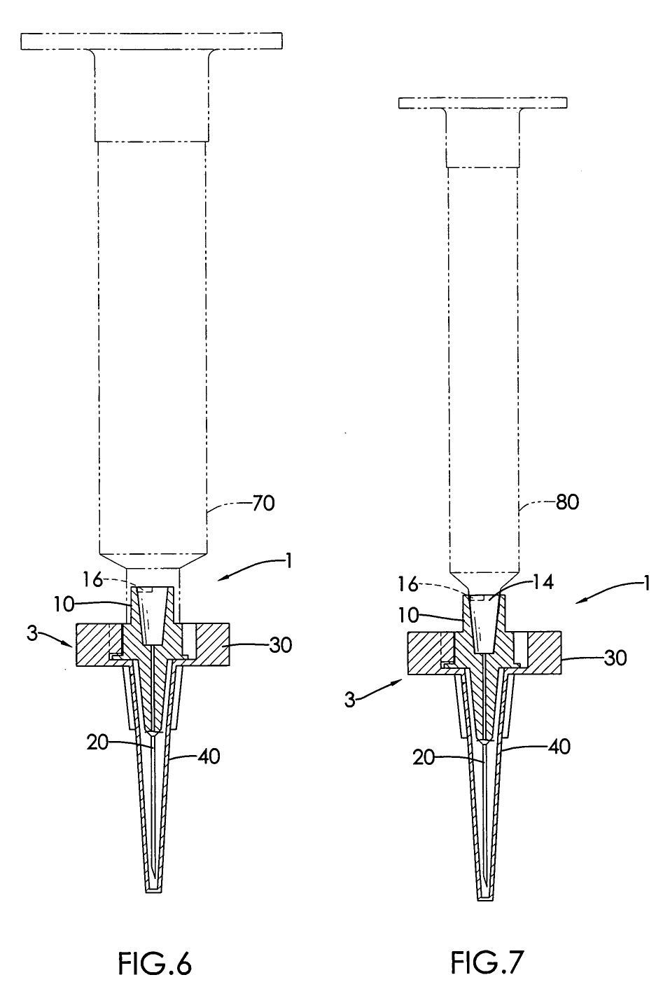

With reference to FIGS. 1, 2, 6 and 7, a needle and cover device is mounted to a large barrel (70) or a small barrel (80) and may be further fabricated with a plunger to form a safety syringe. Each of the large and small barrels (70, 80) has a bottom mounting tube. The bottom mounting tube of the large barrel (70) has an inner thread.

The needle and cover device comprises a needle member (1) and a cover member (3).

The needle member (1) is mounted detachably on the bottom mounting tube of the large or small barrel (70, 80) and has a needle hub (10) and a needle (20).

The needle hub (10) is mounted detachably on the bottom mounting tube and has a cylinder (11), multiple spokes (12) and a needle mount (17).

The cylinder (11) is hollow, may be mounted in the bottom mounting tube of the large barrel (70) and has a top end, a bottom end and a tapered central hole (14) and may further have two engaging tabs (16). The tapered central hole (14) is defined through the cylinder (11), may be mounted around the bottom mounting tube of the small barrel (80). The engaging tabs (16) are formed on and protrude radially out from the cylinder (11) and serve as an outer thread engaged with the inner thread of the bottom mounting tube of the large barrel (70).

The spokes (12) are formed on and protrude transversely out from the cylinder (11) adjacent to the bottom end and each spoke (12) has a locking recess (13) and a limiting tab (15). The locking recess (13) is defined transversely in the spoke (12) and may have a tangent surface (131) and a normal surface (132). The normal surface (132) is substantially perpendicular to the tangent surface (131). The limiting tab (15) is formed on and protrudes transversely out from the spoke (12) adjacent to the locking recess (13) at the bottom end.

The needle mount (17) is tapered, is formed on and protrudes axially from the bottom end of the cylinder (11) and has a through hole defined axially through the needle mount (17) and communicating with the tapered central hole (14).

The needle (20) is mounted in the through hole of the needle mount (17).

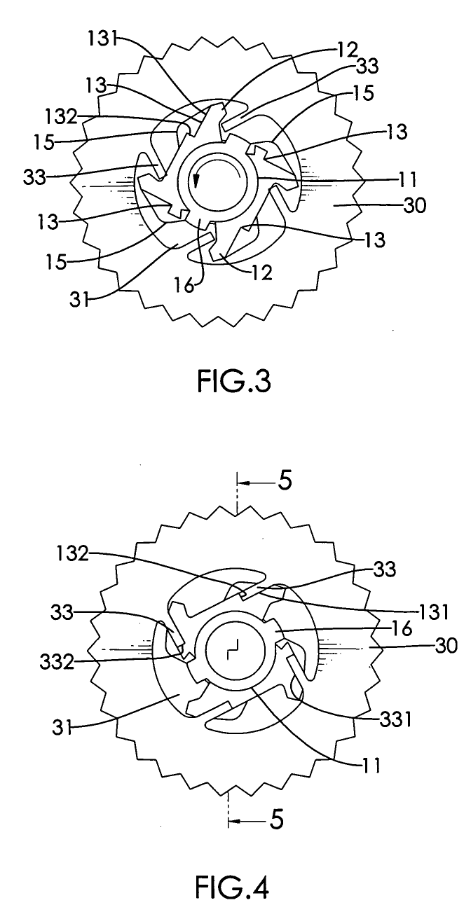

With further reference to FIGS. 3-5, the cover member (3) detachably covers the needle member (1) and is capable of locking and holding the needle member (1) so that removing the cover member (3) detaches the needle member (1) from the large or small barrel (70, 80). The cover member (3) has a locking ring (30) and a cover (40).

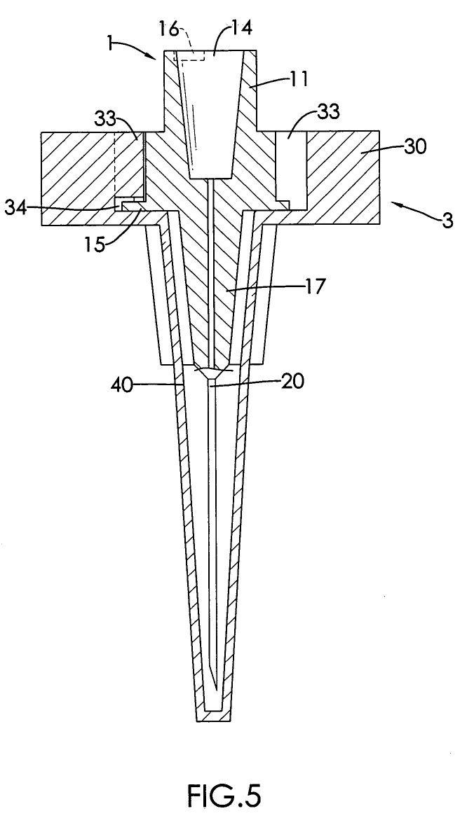

The locking ring (30) selectively locks and holds the needle hub (10) and has a central hole (31), an annular inside surface, an annular inner bottom surface, multiple locking protrusions (33), and multiple gaps (34). The central hole (31) is defined axially through the locking ring (30). The locking protrusions (33) are formed on and protrude transversely inward from the annular inside surface of the locking ring (30) and correspond respectively to and are selectively engaged respectively with the locking recesses (13) in the needle hub (10). Each locking protrusion (33) may have a side surface (331) and an end surface (332), as shown in FIG. 4. The side surface (331) selectively abuts the tangent surface (131) of a corresponding locking recess (13) to prevent the needle hub (10) from rotating counterclockwise relative to the locking ring (30). The end surface (332) selectively abuts the normal surface (132) of the locking recess (13) to prevent the needle hub (10) from rotating clockwise relative to the locking ring (30). The gaps (34) correspond respectively to the locking protrusions (33), are defined respectively between the locking protrusions (33) and the annular inner bottom surface and selectively hold the limiting tabs (15) respectively, as shown in FIG. 5.

When the locking ring (30) locks the needle hub (10), the locking protrusions (33) are engaged respectively with the locking recesses (13) to prevent the needle hub (10) from rotating. Also, the limiting tabs (15) are engaged respectively with the gap (34) and are aligned respectively with the locking protrusions (33) so that the locking protrusions (33) block the limiting tabs (15) to prevent the needle hub (10) from moving axially.

The cover (40) is formed and protrudes axially from the locking ring (30) and has a cavity defined in the cover (40), communicating with the central hole of the locking ring (30) and holding the needle (20).

When the needle (20) is used and has to be disposed, the needle member (1) extends in the cover member (3), as shown in FIG. 3. Then the needle hub (10) is rotated counterclockwise to engage the locking protrusions (33) of the locking ring (30) respectively with the locking recesses (13) of the needle hub (10), as shown in FIGS. 4 and 5. Because the limiting tabs (15) are blocked by the locking protrusions (33), pulling or unscrewing the cover member (3) detaches the needle member (1) from the large or small barrel (70, 80).

Removing the used needle member (1) only needs two simple steps, extending the needle member (1) into the cover member (3) and then rotating needle member (1) or the cover member (3). The removed needle member (1) is locked and held in the cover member (3) to prevent nurses, doctors and workers dealing with the discards needle member (1) from being injured by the needle (20). Therefore, needle and cover device is convenient in use and safe.

Even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms in which the appended claims are expressed.

1. A needle and safety cover device for a syringe comprising:
a needle member attachable to an otherwise conventional syringe and having a needle hub a cylinder being hollow and having a top end, a bottom end and a tapered central hole defined through the cylinder;
multiple spokes formed on and protruding transversely out from the cylinder adjacent to the bottom end and each spoke having a locking recess defined transversely in the spoke; and
a limiting tab formed on and protruding transversely out from the spoke adjacent to the locking recess at the bottom end; and
needle mount formed on and protruding axially from the bottom end of the cylinder and having a through hole defined axially through the needle mount and communicating with the tapered central bole; and
needle mounted in the through hole of the needle mount; and
a cover member detachably covering the needle member, being capable of locking and holding the needle member and having a locking ring selectively locking and holding the needle hub and having a central hole and a grippable outer circumference;
an annular inside surface;
an annular inner bottom surface;
multiple locking protrusions formed on and protruding transversely inward from the annular inside surface of the locking ring and corresponding respectively to and selectively engaged respectively with the locking recesses in the needle hub;
multiple gaps corresponding respectively to the locking protrusions, are defined respectively between the locking protrusions and the annular inner bottom surface and selectively holding the limiting tabs respectively, wherein the gaps respectively hold the limiting tabs when the locking protrusions are engaged respectively with the locking recesses; and
cover formed and protruding axially from the locking ring and having a cavity defined in the cover, communicating with the central hole of the locking ring and holding the needle;
wherein when said cover member is rotatably engaged with said needle member said cover member and said needle member are locked together to safely prevent accidental access to said needle.

2. The needle and cover device as claimed in claim 1, wherein:
each locking recess bas a tangent surface and a normal surface substantially perpendicular to the tangent surface; and
each locking protrusion has a side surface selectively abutting the tangent surface of a corresponding locking recess and a end surface selectively abutting the normal surface of the locking recess.

3. The needle and cover device as claimed in claim 2, wherein the cylinder further has two engaging tabs formed on and protruding radially out from the cylinder.
